(19) United States
(12) Patent Application Publication
(10) Pub. No.: US 2026/0327180 A1
(43) Pub. Date: Oct. 22, 2026

(54) TRANSESTERIFICATION PROCESS

(71) Applicant: Firmenich SA, Satigny (CH)

(72) Inventor: Anatoly ALEXANDER, Plainsboro, NJ (US)

(21) Appl. No.: 19/113,993

(22) PCT Filed: Sep. 21, 2023
(86) PCT No.: PCT/EP2023/076018

§ 371 Date: Mar. 21, 2025

Related U.S. Application Data
(60) Provisional application No. 63/409,510, filed on Sep. 23, 2022.

(30) Foreign Application Priority Data
Oct. 21, 2022 (EP) 22202899.5

(51) Int. Cl. C07C 67/02; B01J 21/04; B01J 23/46

(52) U.S. Cl. CPC C07C 67/02; B01J 21/04; B01J 23/462

(57) ABSTRACT

Disclosed herein is a continuous process for the transesterification of a compound including at least one C₁₋₄ carboxylic ester group in a presence of a C₁₋₄ alcohol and a transesterification catalyst where the transesterification step is performed in at least two subsequent reactors separated by a separation unit. Also disclosed herein is a process for preparing diethyl 1,4-cyclohexanedicarboxylate including a transesterification step of dimethyl terephthalate in the presence of ethanol and a reduction step of diethyl terephthalate.

### TECHNICAL FIELD

The present invention relates to the field of organic synthesis. More particularly, it provides a continuous process for the transesterification of a compound comprising at least one C₁₋₄ carboxylic ester group in a presence of a C₁₋₄ alcohol and a transesterification catalyst wherein the transesterification step is performed in at least two subsequent reactors separated by a separation unit. The invention further relates to a process for preparing diethyl 1,4-cyclohexanedicarboxylate comprising a transesterification step of dimethyl terephthalate in the presence of ethanol and a reduction step of diethyl terephthalate.

### BACKGROUND

The access to compounds comprising carboxylic ester group is highly sought as they represent highly desirable skeletons which could be used as such or as key intermediates useful to prepare more complex compounds in different fields such as, among others, perfumery, cosmetic, pharmaceutic or agrochemistry. A multitude of methodologies has been developed in this context such as transesterification. Said reaction could be quite challenging to implement in large scale as requesting long reaction times and large energy use while suffering low productivity.

Being a key reaction use to produce valuable compounds, there is always a need for new processes showing an improved yield and selectivity and reducing the energy consumption.

The present invention provides a solution to the above problem by performing the transesterification under continuous conditions in at least two subsequent reactors separated by a separation unit wherein the alcohol formed during the process is removed. To the best of our knowledge, in the prior art there is no report of such a continuous process as disclosed in the present invention.

### BRIEF DESCRIPTION OF THE DRAWINGS

FIG. 1 is a schematic process flow sheet illustrating an embodiment of the invention.

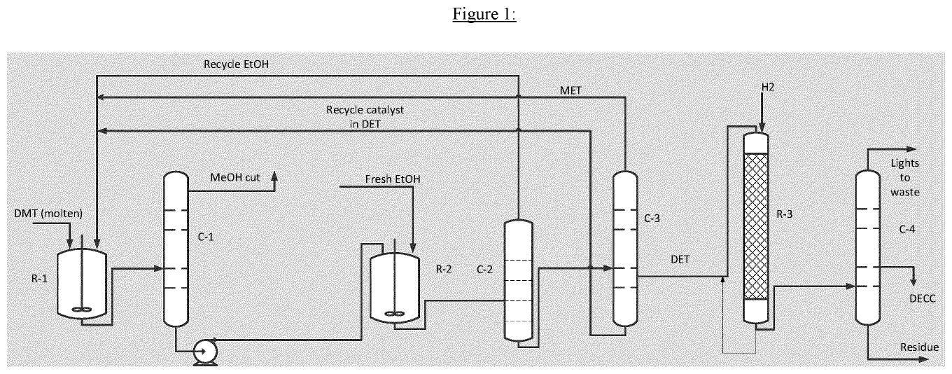

### DESCRIPTION OF THE INVENTION

Surprisingly, it has now been discovered that performing the transesterification process in a conditions mode in at least 2 subsequent reactors separated by a separation unit allows producing a significant higher amount of desired product in a less time leading to reduce the energy consumption.

Therefore, the first object of the present invention is a continuous process for the transesterification of a compound comprising at least one C₁₋₄ carboxylic ester group in a presence of a C₁₋₄ alcohol and a transesterification catalyst wherein the transesterification step is performed in at least two subsequent reactors separated by a separation unit. In other words, the first object of the present invention is a continuous process for the transesterification of a compound comprising at least one C₁₋₄ carboxylic ester group wherein the compound comprising at least one C₁₋₄ carboxylic ester group is reacted with a C₁₋₄ alcohol in a presence of a transesterification catalyst and wherein the transesterification step is performed in at least two subsequent reactors separated by a separation unit.

For the sake of clarity, it is understood that, by the expression “transesterification”, it is intended the usual meaning in the art, i.e. that the starting material and the produced compound comprise at least one carboxylic ester group. In other words, the transesterification is, for example, the preparation of a compound of formula R—COO—R² starting from a compound of formula R—COO—R¹ wherein R¹ and R² groups are different. During said process, an alcohol of formula HOR¹ is also formed being different than the C₁₋₄ alcohol used in the invention's process.

The term “a compound comprising at least one C₁₋₄ carboxylic ester group” is understood as a compound comprising at least a group of formula —COO—R1 wherein R¹ is a C₁₋₄ alkyl group. In other words, the compound comprising at least one C₁₋₄ carboxylic ester group is a compound of formula

R—COO—R¹ (I)

wherein R¹ is a C₁₋₄ alkyl group and R is a C₁₋₁₈ hydrocarbon group optionally comprising one or more C₁₋₄ carboxylic ester groups.

It is understood that by “ . . . hydrocarbon group . . . ” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynyl group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

The term “optionally” is understood that the R group can or cannot comprise a certain functional group. The term “one or more” is understood as comprising 1 to 7, preferably 1 to 5, preferably 1 to 3 and more preferably 1 to 2 of a C₁₋₄ carboxylic ester group.

The term “C₁₋₄ alcohol” is understood as an alcohol of formula R²OH wherein R² is a C₁₋₄ alkyl group. The C₁₋₄ alcohol comprises only one hydroxy group and does not comprise any other functional group; i.e. the C₁₋₄ alcohol is a mono alcohol. The C₁₋₄ alcohol is not glycerine or a fatty alcohol.

The term “alkyl” is understood as comprising branched and linear alkyl group.

The term “separation unit” is understood as any means allowing to separate molecules from a reaction mixture comprising several molecules, in particular to separate molecules having a different boiling points. Non-limiting examples of suitable separation unit may include distillation column, rectification device, a membrane, a pervaporation device, an adsorption unit, an absorption unit.

According to one embodiment of the invention, the compound comprising at least one C₁₋₄ carboxylic ester group is not a fatty acid ester, a monoglycerides, a diglycerides, or a triglycerides.

According to one embodiment of the invention, the compound comprising at least one C₁₋₄ carboxylic ester group is a diester compound. Particularly the diester is a compound of formula

R¹—OOC—R′—COO—R¹ (II)

wherein both R¹, independently from each other, is a C₁₋₄ alkyl group and R′ is a C₁₋₁₈hydrocarbon group optionally comprising one or more C₁₋₄ carboxylic ester groups.

According to any embodiment of the invention, R¹ may be a C₁₋₃ alkyl group, particularly, a methyl or ethyl group, more particularly, a methyl group. In other words, the carboxylic ester group may be a C₁₋₃ carboxylic ester group and the alcohol formed/released during the invention' process may be a C₁₋₃ alcohol, particularly a C₁₋₂ carboxylic ester group and a C₁₋₂ alcohol, even more particularly, a C₁ carboxylic ester group and methanol. Particularly, the alcohol formed/released during the invention' process is not glycerine or ethylene glycol.

According to any embodiment of the invention, R may be a C₁₋₁₆ hydrocarbon group optionally comprising one or more C₁₋₄ carboxylic ester groups. Particularly, R may be a C₁₋₁₄ hydrocarbon group optionally comprising one or more C₁₋₄ carboxylic ester groups. Particularly, R may be a C₁₋₁₂ hydrocarbon group optionally comprising one or more C₁₋₄ carboxylic ester groups. Particularly, R may be a C₁₋₁₀ hydrocarbon group optionally comprising one or more C₁₋₄ carboxylic ester groups. Particularly, R may be a C₁₋₈ hydrocarbon group optionally comprising one or more C₁₋₄ carboxylic ester groups. Particularly, R may be a C₁₋₈ alkyl, a C₃₋₈ cycloalkyl, a C₂₋₈ alkenyl group or a phenyl group; each optionally substituted by one or more C₁₋₄ carboxylic ester groups. Particularly, R may be a C₁₋₆ alkyl, a C₃₋₆ cycloalkyl, a C₂₋₆ alkenyl group or a phenyl group; each optionally substituted by one or more C₁₋₄ carboxylic ester groups. Particularly, R may be a C₁₋₆ alkyl, a C₃₋₆ cycloalkyl, a C₂₋₆ alkenyl group or a phenyl group; each optionally substituted by one or two C₁₋₄ carboxylic ester groups. Particularly, R may be a C₁₋₆ alkyl, a C₃₋₆ cycloalkyl, a C₂₋₆ alkenyl group or a phenyl group optionally substituted by one C₁₋₄ carboxylic ester group. Even more particularly, R may be a C₁₋₆ alkyl, a C₅₋₆ cycloalkyl, a C₂₋₆ alkenyl group or a phenyl group optionally substituted by one C₁₋₄ carboxylic ester group.

According to any embodiment of the invention, and R′ may be a C₁₋₁₆ hydrocarbon group. Particularly, R′ may be a C₁₋₁₄ hydrocarbon group. Particularly, R′ may be a C₁₋₁₂ hydrocarbon group. Particularly, R′ may be a C₁₋₁₀ hydrocarbon group. Particularly, R′ may be a C₁₋₈ hydrocarbon group. Particularly, R′ may be a C₁₋₈ alkanediyl, a C₃₋₈ cycloalkanediyl, a C₂₋₈ alkenediyl group or a phenylene group. Particularly, R′ may be a C₁₋₆ alkanediyl, a C₃₋₆ cycloalkanediyl, a C₂₋₆ alkenediyl group or a phenylene group. Particularly, R′ may be a C₁₋₆ alkanediyl, a C₅₋₆ cycloalkanediyl, a C₂₋₆ alkenediyl group or a phenylene group. Even more particularly, R′ may be a 1,4-phenylene group.

According to any embodiment of the invention, the C₁₋₄ alcohol may be a C₂₋₃ alcohol, preferably ethanol.

According to any embodiment of the invention, the released alcohol from the compound comprising at least one C₁₋₄ carboxylic ester; i.e. the compound of formula R'OH, has a lower boiling point than the C₁₋₄ alcohol; i.e. compound of formula R²OH. Particularly, the difference between the boiling point of the C₁₋₄ alcohol; i.e. compound of formula R2OH and the boiling point of the released alcohol from the compound comprising at least one C₁₋₄ carboxylic ester; i.e. the compound of formula R¹OH, is comprised between 10 and 30 °C, particularly, between 10 and 20 °C, even more particularly, between 12 and 18 °C.

According to any embodiment of the invention, the compound comprising at least one C₁₋₄ carboxylic ester group is dimethyl terephthalate or dimethyl 1,4-cyclohexanedicarboxylate.

According to any embodiment of the invention, the released alcohol from the compound comprising at least one C₁₋₄ carboxylic ester group is totally or partly removed in the separation unit and the C₁₋₄ alcohol is added in each reactor. Particularly, the C₁₋₄ alcohol added in each reactor is the same; i.e. in each reactor ethanol is added.

According to any embodiment of the invention, the reactors used in the invention process may be any reactors suitable for continuous process. The reactors may be identical or different. Non-limiting examples of suitable reactor may include plug-flow reactor, continuously stirred tank reactor, Laminar flow reactor, loop reactor, micro reactor, reactor divided into a plurality of sections and a combination thereof. Particularly, the reactors may be plug-flow reactor, continuously stirred tank reactor and a combination thereof.

According to any embodiment of the invention, the continuous process is performed in two reactors. The residence time in each reactor will depend of the type of reactors. The residence time may be comprised between 0.01 hrs and 100 hrs, particularly between 0.2 hrs and 20 hrs.

According to any embodiment of the invention, the separation unit may be a distillation column. Said distillation column used in the invention process may be any distillation column suitable for continuous process. The distillation column may comprise plates or trays or packing material. A person skilled in the art is able to select and sized the distillation column as a function of the melting and boiling point of the starting and final products.

According to any embodiment of the invention, the distillation may be carried out at atmospheric pressure or at reduced pressure, in particular at a pressure of less than 200×10⁵ Pa (200 bars), for example at a pressure comprised between 5×10⁵ Pa and 100×10⁵ Pa (5 and 100 bars).

According to any embodiment of the invention, the invention's process comprises
a) the reaction of a transesterification of a compound comprising at least one C₁₋₄ carboxylic ester group with a C₁₋₄ alcohol in a presence of a transesterification catalyst in a first reactor;
b) then the reaction mixture of step a) is distilled in a distillation column wherein the released alcohol is totally or partly removed; and
c) then the reaction mixture is flowed in a second reactor wherein the C₁₋₄ alcohol is added.

According to any embodiment of the invention, the C₁₋₄ alcohol added in step a) and in step c) is identical.

According to any embodiment of the invention, the second reactor is followed by at least one separation unit wherein the unreacted or partially reacted compound comprising at least one C₁₋₄ carboxylic ester group, the released alcohol, the excess added C₁₋₄ alcohol and the transterification catalyst are removed. Particularly, the second reactor is followed by two separation units. Said separation units are distillation column.

According to any embodiment of the invention, the transesterification catalyst is a Lewis acid, a Bronsted acid or a base, particularly a Lewis acid. Specific and non-limiting examples of transesterification catalyst may be selected from the group consisting of tinorganic or titaniumorganic compounds, in situ formed tinorganic compounds via reaction of dialkyltin oxides with acid of the ester being transesterified, preferably dialkyltin derivatives such as oxides (C₄H₉)₂SnO, (C₈H₁₇)₂SnO, dicarboxylates such as dibutyltin dilaurate, dioctyltin dicarboxylate and mixtures thereof.

The transesterification catalyst can be added into the reaction medium of the invention's process in a large range of concentrations. As non-limiting examples, one can cite as acid concentration values those ranging from about 0.001 to about 5 mol %, relative to the amount of the compound comprising at least one C₁₋₄ carboxylic ester, preferably from 0.02 to about 0.5 mol %, relative to the amount of the compound comprising at least one C₁₋₄ carboxylic ester. The optimum concentration of the transesterification catalyst will depend, as the person skilled in the art knows, on the nature of the latter, on the nature of the compound comprising at least one C₁₋₄ carboxylic ester, on the nature of the C₁₋₄ alcohol, on the reaction temperature as well as on the flow of the process.

The C₁₋₄ alcohol can be added into the reaction medium of the invention's process in a large range of concentrations. As non-limiting examples, one can cite as C₁₋₄ alcohol values those ranging from about 2 to about 20 equivalents, relative to the amount of the compound comprising at least one C₁₋₄ carboxylic ester, preferably at least 5 equivalents, relative to the amount of the compound comprising at least one C₁₋₄ carboxylic ester. The optimum concentration of the C₁₋₄ alcohol will depend, as the person skilled in the art knows, on the nature of the latter, on the nature of the compound comprising at least one C₁₋₄ carboxylic ester, on the nature of the transesterification catalyst, on the reaction temperature as well as on the flow of the process.

According to any one of the invention's embodiments, the C₁₋₄ alcohol is added in each reactor of the invention's process, in particular, the same C₁₋₄ alcohol is added in each reactor.

According to any one of the invention's embodiments, the invention's process is carried out at a temperature comprised between 20 °C and 250 °C. In particular, the temperature is in the range between 70 °C and 200 °C. Of course, a person skilled in the art is also able to select the preferred temperature as a function of the melting and boiling point of the starting and final products as well as the desired time of reaction or conversion.

The invention's process may be performed at a pressure comprised between 0.1×10⁵ Pa and 100×10⁵ Pa (0.1 to 100 bars) or even more if desired. Again, a person skilled in the art is well able to adjust the pressure as a function of the catalyst load and of the compound comprising at least one C₁₋₄ carboxylic ester. As examples, one can cite typical pressures of 1 to 50×10⁵ Pa (1 to 50 bars).

The invention's process may be performed under inert atmosphere such as nitrogen and/or argon.

The invention's process can be carried out in the presence or absence of a solvent. When a solvent is required or used for practical reasons, then any solvent current in such reaction type can be used for the purposes of the invention. The choice of the solvent is function of the nature of the compound comprising at least one C₁₋₄ carboxylic ester group and/or catalyst and the person skilled in the art is well able to select the solvent most suitable in each case to optimize the reaction.

According to any one of the invention's embodiments, the C₁₋₄ alcohol and the transesterification catalyst are recycled.

The invention's process for compound comprising more than one C₁₋₄ carboxylic ester involves the formation of mono transesterified compound as an intermediate; such intermediate may be present at a certain level in the final reaction mix, and may be recycled in the invention's process. In addition, unreacted starting materials may be also recycled in the invention's process.

A second object of the present invention is the use of the process as defined above to produce perfumery, cosmetic or pharmaceutic products, particularly, perfumery products. Particularly, the perfumery product may be diethyl 1,4-cyclohexanedicarboxylate.

A further object of the present invention is a process for preparing diethyl 1,4-cyclohexanedicarboxylate comprising the step of
a) the transesterification as defined above of dimethyl terephthalate in the presence of ethanol providing diethyl terephthalate; and
b) the reduction of diethyl terephthalate obtained in step a).

In other words, a further object of the present invention is a process for preparing diethyl 1,4-cyclohexanedicarboxylate comprising the step of
a) the transesterification as defined above wherein dimethyl terephthalate reacts with ethanol in the presence of a transesterification catalyst providing diethyl terephthalate; and
b) the reduction of diethyl terephthalate obtained in step a).

According to any one of the invention's embodiments, the reduction is a hydrogenation using molecular H₂ and a hydrogenation catalyst. The hydrogenation catalyst may be a metal in elemental metallic form, in particular palladium or ruthenium in elemental metallic form.

According to any one of the above embodiments of the invention, said palladium (Pd) or Ruthenium (Ru) is supported on a carrying material.

For the sake of clarity, by carrying material it is intended a material wherein it is possible to deposit such metal and which is inert toward the substrate (diethyl terephthalate).

According to any one of the above embodiments of the invention, specific and non-limiting examples of carrying material is carbon, silica or aluminum oxide. Such supports are well known to a person skilled in the art.

The supported palladium (Pd) or ruthenium (Ru) catalysts are known compounds and are commercially available. A person skilled in the art is able to select the preferred kind of metal as the way that it was deposit on the support, as the proportion of metal on support material, as the form (powder, granules, pellets, extrudates, mousses . . . ) and as the surface area of the support. Particularly, the hydrogenation catalyst is alumina supported ruthenium.

According to any one of the above embodiments of the invention, the amount of metal relative to the support can range between 0.05% and 25% w/w, or even between 0.4% and 6%, relative to the weight on the support used.

The hydrogenation catalyst can be added into the reaction medium of the invention's process in a large range of concentrations. As non-limiting examples, one can cite as metal concentration values those ranging from 0.001 mol % to 1mol %, relative to the total amount of diethyl terephthalate. Preferably, the metal concentration will be comprised between 0.02 mol % to 1mol %, or even between 0.04 mol % to 1mol %. It goes without saying that the optimum concentration of metal will depend, as the person skilled in the art knows, on the nature of the latter, if the process is run in batch or continuously, on the temperature and on the pressure of H₂ used during the process, as well as the desired time of reaction.

The hydrogenation catalyst may be recycled at the end of the invention's process. In other words, the hydrogenation catalyst may be recovered at the end of the invention's process and use several times in the invention's process.

According to any one of the above embodiments of the invention, the molecular hydrogen can be used pure or mixed with an inert gas. Specific and non-limiting examples of such inert gas are nitrogen or argon. When the molecular hydrogen is used in combination with an inert gas, the H₂/inert gas volume ratio is comprised between 1/1 to 0.01/1 and more preferably the ratio is 0.05/1.

The molecular hydrogen can be added into the reaction medium of the invention's process in a large range of ratios relative to the substrate. As non-limiting examples, one can cite as molecular hydrogen ratio values those ranging from 100 mol % to 5000 mol %, relative to the amount of diethyl terephthalate. Even more preferably, the molecular hydrogen concentration will be comprised between 300 mol % to 2000 mol % relative to the amount of diethyl terephthalate. Of course, a person skilled in the art is well able to adjust the pressure or the flow (e.g. in a continuous process) of molecular hydrogen to obtain this range of concentration as a function of the process is batch or continuous. The person skilled in the art is also well able to adjust the concentration of molecular hydrogen as a function of the catalyst load and of dilution of diethyl terephthalate in the solvent.

The reduction can be carried out under batch or continuous conditions. According to a particular embodiment of the invention, the reduction is a continuous one, as it allows higher productivity.

The reduction can be carried out in the presence or absence of a solvent. When a solvent is required or used for practical reasons, then any solvent current in such reaction type can be used for the purposes of the invention. Non-limiting examples include C₆₋₁₂ aromatic solvents such as toluene, 1,3-diisopropylbenzene, p-cymene, cumene, pseudocumene, benzyl acetate, xylene or a mixture thereof, C₃₋₁₆ alkane such as hexadecane, ether solvents such as tetrahydrofuran, butyl ether, methyltetrahydrofuran or a mixture thereof, esters such as ethyl acetate or diethyl cyclohexyldicarboxylate(reaction product), the latter being the preferred solvent. The choice of the solvent is a function of the hydrogenation catalyst and the person skilled in the art is well able to select the solvent most convenient in each case to optimize the reaction.

The temperature at which the reduction can be carried out is comprised between 90 °C and 300 °C. More preferably in the range of between 100 °C and 200 °C for a continuous process. Of course, a person skilled in the art is also able to select the preferred temperature as a function of the melting and boiling point of the starting and final products as well as the desired time of reaction or conversion.

The reduction may be performed at a pressure comprised between 0.1×10⁵ Pa and 100×10⁵ Pa (0.1 to 100 bars) or even more if desired. Again, a person skilled in the art is well able to adjust the pressure as a function of the catalyst load and the desired time of reaction or conversion. As examples, one can cite typical pressures of 1 to 50×10⁵ Pa (1 to 50 bars).

According to any one of the above embodiments of the invention, the reduction is performed in a fix-bed reactor.

According to any one of the above embodiments of the invention, the reduction is performed in the absence of transesterification catalyst.

A further object of the present invention is a process for preparing diethyl 1,4-cyclohexanedicarboxylate comprising the step of
a) the reduction of dimethyl terephthalate; and
b) the transesterification as defined above of dimethyl 1,4-cyclohexanedicarboxylate obtained in step a) in the presence of ethanol providing diethyl 1,4-cyclohexanedicarboxylate.

Typical manners to execute the invention's process are reported herein below in the examples with reference to the accompanying drawing.

### EXAMPLES

The invention will now be described in further detail by way of the following examples, wherein the abbreviations have the usual meaning in the art, the temperatures are indicated in degrees centigrade (°C). The drawing is for an illustration purpose only and the present invention should not be understood to be limited to the precise arrangements and items of equipment as shown in the drawing. In addition the person skilled in the art is well aware that further items of equipment, not represented in the drawing, may be needed to implement the invention process, such as, for example, vacuum pumps, sensors to measure temperature or pressure, valves, etc. . . .

### Example 1

Preparation of Diethyl 1,4-cyclohexanedicarboxylate Following the Invention's Continuous Process

Into a 4,000 gal. (~15 cu.m) pressure reactor R-1 equipped with the agitator and heating system, in a steady mode had been introduced (per hour) 450 kg of molten dimethylterephthalate (DMT), 45 kg recycle solution of tin transesterification catalyst in diethylterephthalate (DET) (from the bottom of C-3) with 2 mol % concentration of the catalyst, 70 kg of the recycle mixture of dimethylterephthalate, methyl ethyl terephthalate (MET) and diethylterephthalate from the top of C-3, and 520 kg of recycle ethanol (purity about 90%) from the overhead of C-2. Temperature in R-1 had been set at 150 °C, pressure at 6 bar, and level of liquid in the reactor was controlled to provide an average residence time of 6 hours.

Product had been continuously withdrawn from R-1, and 70 kg/hr of methanol-rich distillate had been removed in a distillation column C-1. The residue from C-1 together with 120 kg/hr of fresh ethanol had been continuously introduced into the reactor R-2 of design similar to R-1 and at the similar conditions (pressure, temperature, residence time). The reaction mix leaving R-2 had been distilled in a column C-2 producing 520 kg/hr of recycle ethanol (to be returned to R-1), and the residue from C-2 had been separated in a column C-3 where 512 kg/hr of 99.3% pure diethylterephthalate were removed as a side draw, and 70 kg/hr of the overhead cut and 45 kg/hr of residue were recycled to R-1.

Diethylterephthalate had been continuously hydrogenated in a trickle-bed catalytic reactor R-3 over the supported precious-metal catalyst at 55 bar and 120 °C with about 75% of the reaction mix returned back to the top of the reactor to avoid undesirably high temperatures due to the exothermicity of hydrogenation. 530 kg/hr of the effluent containing about 90% of diethyl 1,4-cyclohexanedicarboxylate (DECC), 6% of unreacted starting material and 4% of the byproducts of hydrogenation had been distilled in a column C-4 resulting in 465 kg/hr of diethyl 1,4-cyclohexanedicarboxylate produced as a side draw stream, and 35 kg/hr of diethyl terephthalate recovered from the bottom and sent to the circulating feed of the reactor R-3.

### Example 2

Preparation of Diethyl 1,4-cyclohexanedicarboxylate Following the Invention's Process

Processing up to the hydrogenation of diethylterephthalate had been done similarly to Example 1, but instead of the continuous hydrogenation in a fixed-bed reactor, diethylterephthalate had been hydrogenated in a high-pressure autoclave at 70bar/140 °C for 12 hours. Reaction mix contained 99.0% 1,4-cyclohexanedicarboxylate, less than 0.05% of unreacted diethylterephthalate, and 1.0% of byproducts.

### Example 3

### Preparation of Diethylterephthalate (Comparative Example)

21,000 kg of dimethylterephthalate flakes and 0.2 mol % of soluble tin catalyst were loaded in a 10,000 gal. (37.5 cu.m) pressure rated reactor equipped with the heating system, agitator and distillation column. Heating had been turned on, and dimethylterephtalate molten down within 18 hours. After that, the pressure had been set up at 3 bar, and 4,000 kg of ethanol had been pumped in. As the reaction byproduct methanol had been distilled out (steam flow to the heater ~500 kg/hr), more fresh ethanol were added-so gradually the distillate composition became enriched with ethanol. After 90 hrs, reaction mix contained (excluding ethanol and methanol) 91% of diethylterephthalate, and about 11,000 kg of alcohol byproduct (about 50% each of methanol and ethanol) had been distilled out. After distillation in a separate still of the material remaining in the reactor, ~17,000 kg of pure diethylterephthalate were obtained which is equivalent to the productivity of 157 kg per hour (including the melting and reaction time but excluding the charging and prep time, etc.).

1. A continuous process for the transesterification of a compound comprising at least one C₁₋₄ carboxylic ester group in a presence of a C₁₋₄ alcohol and a transesterification catalyst, wherein the continuous process comprises a transesterification step, wherein the transesterification step is performed in at least two subsequent reactors separated by a separation unit.

2. The continuous process according to claim 1, wherein the released alcohol from the compound comprising at least one C₁₋₄ carboxylic ester group is totally or partly removed in the separation unit and the C₁₋₄ alcohol is added in each reactor.

3. The continuous process according to claim 1, wherein the continuous process is performed in two reactors.

4. The continuous process according to claim 1, wherein the second reactor is followed by one or two separation units.

5. The continuous process according to claim 1, wherein the separation units are distillation columns.

6. The continuous process according to claim 1, wherein the transesterification catalyst is a Lewis acid, a Bronsted acid or a base.

7. The continuous process according to claim 1, wherein the transesterification catalyst is selected from the group consisting of tinorganic or titaniumorganic compounds, in situ formed tinorganic compounds via reaction of dialkyltin oxides with acid of the ester being transesterified, (C₄H₉)₂SnO, (C₈H₁₇)₂SnO, dibutyltin dilaurate, dioctyltin dicarboxylate and mixtures thereof.

8. The continuous process according to claim 1, wherein the compound comprising at least one C₁₋₄ carboxylic ester group is a diester compound.

9. The continuous process according to claim 1, wherein the carboxylic ester group is a C₁ carboxylic ester group.

10. The continuous process according to claim 1, wherein the compound comprising at least one C₁₋₄ carboxylic ester group is dimethyl terephthalate.

11. The continuous process according to claim 1, wherein the alcohol is a C₂₋₃ alcohol.

12. A process for preparing diethyl 1,4-cyclohexanedicarboxylate comprising:
a) transesterifying dimethyl terephthalate in the presence of ethanol in at least two subsequent reactors separated by a separation unit, thereby providing diethyl terephthalate; and
b) reducing the diethyl terephthalate obtained in step a).

13. The process according to claim 12, wherein the reduction is carried out under continuous conditions.

14. The process according to claim 12, wherein the reducing is a hydrogenating using molecular H₂ and a hydrogenation catalyst.

15. The process according to claim 14, wherein the hydrogenation catalyst is selected from the group consisting of supported Ru or Pd metals.

16. The continuous process according to claim 1, wherein the alcohol is ethanol.

17. The process according to claim 14, wherein the hydrogenation catalyst is alumina supported ruthenium.
